(19) United States
(12) Patent Application Publication
(10) Pub. No.: US 2025/0120993 A1
(43) Pub. Date: Apr. 17, 2025

(54) COMPOSITION FOR CONTROLLING BLOOD SUGAR COMPRISING GALLOCATECHIN GALLATE AND ISOQUERCITRIN

(71) Applicant: AMOREPACIFIC CORPORATION, Seoul (KR)

(72) Inventors: Hyun Woo JEONG, Yongin-si (KR); Wanki KIM, Yongin-si (KR); Jong Hwa ROH, Yongin-si (KR)

(73) Assignee: AMOREPACIFIC CORPORATION, Seoul (KR)

(21) Appl. No.: 18/883,178

(22) Filed: Sep. 12, 2024

(30) Foreign Application Priority Data
Oct. 11, 2023 (KR) 10-2023-0135349

(51) Int. Cl. A61K 31/7048; A61K 31/353; A61P 3/08

(52) U.S. Cl. CPC A61K 31/7048; A61K 31/353; A61P 3/08

(57) ABSTRACT

A method for controlling blood sugar, including administering an effective amount of Gallocatechin Gallate (GCG) and Isoquercitrin (IQC) to a subject in need thereof is disclosed. In an aspect, a composition including a complex component of Gallocatechin Gallate and Isoquercitrin can effectively inhibit gluconeogenesis in liver cells and effectively improve transport of glucose in differentiated fat cells and liver cells, thereby exhibiting excellent blood sugar control efficacy.

### CROSS REFERENCE TO RELATED APPLICATION

The present application claims the priority of Korean Patent Application No. 10-2023-0135349, filed on Oct. 11, 2023, the entire contents of which are hereby incorporated by this reference.

### BACKGROUND OF THE INVENTION

### Field of the Invention

The present specification discloses a composition having an excellent blood sugar control ability.

### Description of the Related Art

Glucose is absorbed into the body through the process of intake, digestion, and uptake of a food, and is also newly produced through gluconeogenesis in liver cells. On the other hand, a concentration of glucose in the blood is reduced due to use as an energy source in cells having active energy metabolism, such as nerve cells and muscle cells, or is reduced by promoting transport of glucose into the cells for the purpose of storing excess energy in fat cells, etc. The blood glucose control is balanced through these various processes, but when the blood glucose control fails for various reasons (for example, fasting blood sugar of 126 mg/dl or more), diabetes occurs. The diabetes is not only a cause of various metabolic diseases such as obesity, hyperlipidemia, and high blood pressure, but it is also a disease that greatly decreases the quality of life because once the diabetes develops, it is difficult to cure and requires lifelong management of eating and lifestyle habits. Accordingly, there is a need to develop health functional foods or medicines for preventing or improve metabolic disease complications and improving the quality of life by suppressing the occurrence of diabetes or improving diabetes symptoms.

### SUMMARY OF THE INVENTION

A purpose of the present disclosure is to provide a composition having an excellent blood sugar control ability.

In order to achieve the above purpose, in an aspect, the present disclosure provides a composition for controlling blood sugar, comprising Gallocatechin Gallate (GCG) and Isoquercitrin (IQC) as an active ingredient.

In an aspect, the composition of the present disclosure comprises a complex component of Gallocatechin Gallate and Isoquercitrin so that gluconeogenesis is effectively inhibited in liver cells and the transport of glucose is effectively promoted in differentiated fat cells and liver cells, thereby making it possible to provide an excellent blood sugar control efficacy.

### BRIEF DESCRIPTION OF THE DRAWINGS

FIG. 1 compares an effect of inhibiting expression of genes related to gluconeogenesis in liver cells according to a composition ratio of Gallocatechin Gallate (GCG) and Isoquercitrin (IQC) mixture (Adiphenon).

FIG. 2 compares an effect of promoting uptake of glucose in fat cells according to a composition ratio of Adiphenon.

FIG. 3 compares an effect of promoting transport of glucose in muscle cells according to a composition ratio of Adiphenon.

FIG. 4 compares a change in expression of citric acid cycle-related genes in fat cells according to a composition ratio of Adiphenon.

FIG. 5 compares a change in expression of citric acid cycle-related genes in muscle cells according to a composition ratio of Adiphenon.

FIG. 6 compares a change in accumulation of lactic acids in cells by treatment with Adiphenon.

FIG. 7 compares an effect of inhibiting expression of gluconeogenesis genes according to a detailed composition ratio of Adiphenon.

FIG. 8 compares an effect of promoting uptake of glucose in fat cells according to a detailed composition ratio of Adiphenon.

FIG. 9 compares an effect of promoting uptake of glucose in muscle cells according to a detailed composition ratio of Adiphenon.

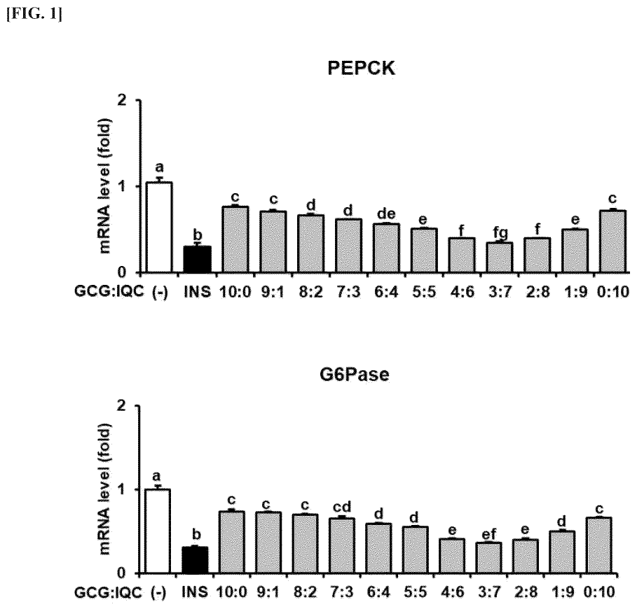

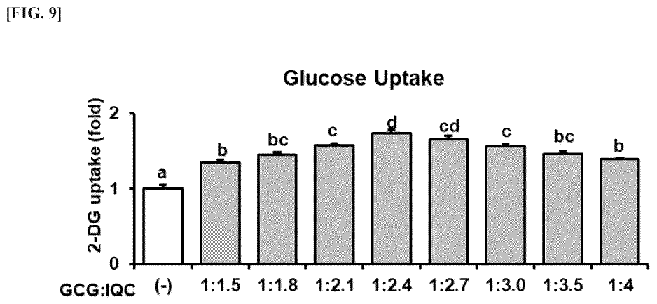

In FIGS. 1 to 9, experimental groups showing statistically significant differences (P<0.05) were indicated with different symbols.

### DETAILED DESCRIPTION OF THE INVENTION

Hereinafter, the present disclosure will be described in detail.

In an aspect, the present disclosure relates to a composition for controlling blood sugar, comprising Gallocatechin Gallate (GCG) and Isoquercitrin (IQC).

In an exemplary embodiment, Gallocatechin Gallate and Isoquercitrin may be contained in a weight ratio of 9:1 or more, 8:2 or more, 7:3 or more, 6:4 or more, 5:5 or more, or 4:6 or more, and may be contained in a weight ratio of 1:9 or less or 2:8 or less. For example, Gallocatechin Gallate and Isoquercitrin may be contained in a weight ratio of 9:1 to 1:9, but is not limited thereto.

In an exemplary embodiment, Gallocatechin Gallate and Isoquercitrin may be contained in a weight ratio of 1:1 or more, 1:1.1 or more, 1:1.2 or more, 1:1.3 or more, 1:1.4 or more, 1:1.5 or more, 1:1.6 or more, 1:1.7 or more, 1:1.8 or more, 1:1.9 or more, 1:2.0 or more, 1:2.1 or more, 1:2.2 or more, or 1:2.3 or more, and may be contained in a weight ratio of 1:9 or less, 1:8 or less, 1:7 or less, 1:6 or less, 1:5 or less, 1:4 or less, 1:3.5 or less, 1:3.4 or less, 1:3.3 or less, 1:3.2 or less, 1:3.1 or less, 1:3 or less, 1:2.9 or less, 1:2.8 or less, 1:2.7 or less, 1:2.6 or less, or 1:2.5 or less. In case Gallocatechin Gallate and Isoquercitrin are contained in the above weight ratio, blood sugar can be effectively controlled. For example, Gallocatechin Gallate and the Isoquercitrin may be contained in a weight ratio of 1:1 to 1:9 or 1:1.5 to 1:4, but is not limited thereto.

In an exemplary embodiment, the blood sugar control may be to lower blood sugar. The active ingredients of the above composition can inhibit decomposition of sugar in the blood or promote cellular uptake of glucose in the blood.

A concentration of glucose in the blood is controlled through a balance between inflow and production of glucose, and consumption and storage of glucose. When glucose concentration is low on an empty stomach, detection of low blood sugar in pancreatic alpha cells causes secretion of glucagon to decompose glycogen stored in liver cells or reversely synthesize glucose from acetyl-coenzyme A. This process is called gluconeogenesis, and this gluconeogenesis is inhibited by insulin secreted during satiety. In case the insulin signaling system is disturbed for some reason and insulin does not perform its role properly despite the presence of insulin (insulin resistance), if gluconeogenesis is not inhibited by insulin, liver cells continue to produce glucose, which may lead to high blood sugar.

In an exemplary embodiment, the composition may be characterized in that it is administered to a subject that needs to decrease gluconeogenesis in liver cells.

In an exemplary embodiment, the composition may be characterized in that it is administered to a subject that needs to reduce expression of gluconeogenesis-related genes, wherein the gluconeogenesis-related genes may be PEPCK (Phosphoenol Pyruvate CarboxyKinase) genes or G6Pase (Glucose 6-Phosphatase) genes.

In an exemplary embodiment, the composition may be administered to a subject that needs to increase uptake and/or transport of glucose in fat cells or liver cells.

In an exemplary embodiment, the composition may be characterized in that it is administered to a subject that needs to increase expression of citric acid cycle-constituting genes, wherein the citric acid cycle-constituting genes may be isocitrate dehydrogenase (IDH) or malate dehydrogenase (MD).

In an exemplary embodiment, the composition may be administered to a subject that needs to inhibit accumulation of lactic acids in cells.

In an exemplary embodiment, the composition may be administered to a subject that needs to promote aerobic energy metabolism in cells.

As used herein, the term “prevention” refers to any action that suppresses or delays diabetes, diabetic complications, or sugar metabolism diseases by administering the present pharmaceutical composition. The term “treatment” refers to any action by which the symptoms of a subject suspected or affected by diabetes, diabetic complications, or sugar metabolism diseases are improved or beneficially changed by administering the present pharmaceutical composition.

As used herein, the term “improvement” refers to any action by which diabetes, diabetic complications, or sugar metabolism diseases are improved or beneficially changed by administering the present composition.

In an exemplary embodiment, the composition may be a pharmaceutical composition for preventing or treating diabetes, diabetic complications, or sugar metabolism diseases.

In an exemplary embodiment, the composition may be a food composition for preventing or improving diabetes, diabetic complications, or sugar metabolism diseases.

In this specification, the diabetes refers to a disease in which a balance between insulin sensitivity and insulin secretion in the body is broken, that is, a disease in which glucose is excreted in the urine due to lack of insulin or decrease in insulin sensitivity, which results in the inability to use sugar in the blood and control the blood sugar. The diabetic complications may be classified into acute complications and chronic complications. Examples of the acute complications may include ketoacidemia, hyperosmotic hyperglycemia, and hypoglycemia, etc., and examples of the chronic complications may include macrovascular complications such as coronary artery disease, cerebrovascular disease and peripheral vascular disease, microvascular complications such as diabetic membrane disease, diabetic nephropathy and diabetic neuropathy, and diabetic foot lesions, etc., but are not limited thereto.

In an exemplary embodiment, a daily dosage of the active ingredient in the composition may be 5 mg/kg or more, 6 mg/kg or more, 7 mg/kg or more, 8 mg/kg or more, 9 mg/kg or more, 10 mg/kg or more, 20 mg/kg or more, 30 mg/kg or more, 40 mg/kg or more, 50 mg/kg or more, 60 mg/kg or more, 70 mg/kg or more, 80 mg/kg or more, 90 mg/kg or more, 100 mg/kg or more, 1 g/kg or more, 2 g/kg or more, 3 g/kg or more, 4 g/kg or more, 5 g/kg or more, or 6 g/kg or more, and may be 60 g/kg or less, 59 g/kg or less, 58 g/kg or less, 57 g/kg or less, 56 g/kg or less, 55 g/kg or less, 54 g/kg or less, 53 g/kg or less, 52 g/kg or less, or 51 g/kg or less. An effect of controlling blood sugar is excellent at the above dosage. If the dosage is less than the above range, the effect of controlling blood sugar is minimal, and if the dosage is higher than the above range, a problem such as toxicity may occur. The above dosage may be administered once or in divided doses several times a day. For example, it may be administered 2 to 24 times per a day, 1 to 2 times per 3 days, 1 to 6 times per a week, 1 to 10 times per 2 weeks, 1 to 15 times per 3 weeks, 1 to 3 times per 4 weeks, or 1 to 12 times per a year, but is not limited thereto.

In an exemplary embodiment, the pharmaceutical composition may be provided in any formulation suitable for topical administration. For example, it may be administered orally, transdermally, intravenously, intramuscularly, or by subcutaneous injection. As an example, the pharmaceutical composition may be an injection, a solution for external use on the skin, a suspension, an emulsion, a gel, a patch, or a spray, but is not limited thereto. The formulation may be easily prepared according to conventional methods well known in the relevant field, and may appropriately contain a surfactant, an excipient, a hydrating agent, an emulsification accelerator, a suspending agent, a salt or buffer for adjusting osmotic pressure, a colorant, a spice, a stabilizer, a preservative, a conserving agent, or other commercially available aids.

In an exemplary embodiment, the active ingredient of the pharmaceutical composition will vary depending on the subject's age, gender, weight, pathological condition and its severity, a route of administration, or judgment of the prescriber. An appropriate dosage which is used based on these factors may be determined within the level of a person skilled in the art.

In an exemplary embodiment, in case the composition is used as an additive for a health functional food, the composition may be added as it is or used together with other foods or food ingredients, and may be used appropriately according to conventional methods. A mixed amount of the active ingredients may be appropriately determined depending on each purpose of use, such as prevention, health, or treatment. A formulation of the health functional food may be in any form of powders, granules, pills, tablets or capsules as well as general foods or beverages.

In an exemplary embodiment, there is no particular limitation on the type of the health functional food, and examples of foods to which the composition can be added include meat, confectionery, noodles, gum, dairy products including ice cream, various soups, beverages, tea, drinks, alcoholic beverages, vitamin complexes, etc., and may include all foods in the conventional sense.

In an exemplary embodiment, in the preparation of the health functional food or beverage, the composition may be added in an amount of 15 parts by weight or less, preferably 10 parts by weight or less, based on 100 parts by weight of the raw material. However, in the case of long-term intake for the purpose of health and hygiene or health control, the above amount may be below said range.

In an exemplary embodiment, the beverages among the health functional foods may contain various flavoring agents or natural carbohydrates as additional ingredients like the typical beverages. The above-mentioned natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. A sweetening agent may include natural sweetening agents such as thaumatin and stevia extract or synthetic sweetening agents such as saccharin and aspartame. A ratio of the natural carbohydrates may be about 0.01 to 0.04 g, preferably about 0.02 to 0.03 g, based on 100 mL of the beverages according to the present disclosure, but are not limited thereto.

In an exemplary embodiment, in addition to the above, the health functional food according to the present disclosure may contain various nutrients, vitamins, electrolytes, flavors, colorants, pectic acids and their salts, alginic acids and their salts, organic acids, protective colloidal thickeners, pH regulators, stabilizers, preservatives, glycerin, alcohol, and carbonating agents used in carbonated beverages. In addition, the health functional food according to the present disclosure may contain fruit flesh for the production of natural fruit juice, fruit juice drinks, and vegetable drinks. These ingredients can be used independently or in combination with each other. A ratio of these additives is not limited, but is generally selected in the range of 0.01 to 0.1 parts by weight based on 100 parts by weight of the health functional food according to the present disclosure.

In other aspect, the present disclosure provides a method for controlling blood sugar, comprising administering to a subject a composition comprising an effective amount of Gallocatechin Gallate (GCG) and Isoquercitrin (IQC).

In another aspect, the present disclosure provides a use of Gallocatechin Gallate (GCG) and Isoquercitrin (IQC) for preparing a composition for controlling blood sugar.

In still another aspect, the present disclosure provides Gallocatechin Gallate (GCG) and Isoquercitrin (IQC) for controlling blood sugar.

In more still another aspect, the present disclosure provides a non-therapeutic use of Gallocatechin Gallate (GCG) and Isoquercitrin (IQC) for controlling blood sugar.

Hereinafter, the constitutions and effects of the present disclosure will be described in more detail through Examples. However, since the following Examples are provided only for illustrative purposes to aid understanding of the present disclosure, the scope and range of the present disclosure should not be limited by them.

### EXAMPLE

### Example 1

### Comparison of an Effect of Inhibiting Gluconeogenesis in Liver Cells by Treatment With a Mixture (Adiphenon) of Gallocatechin Gallate (GCG) and Isoquercitrin (IQC)

The present inventors have confirmed whether a mixture (Adiphenon) of Gallocatechin Gallate (GCG) and Isoquercitrin (IQC) can inhibit gluconeogenesis in liver cells. Specifically, HepG2 cells purchased from ATCC were cultured in a Roswell Park Memorial Institure-1640 medium (Sigma Aldrich) containing 10% fetal bovine serum (Gibco) and 1% penicillin/streptomycin (P/S; Sigma Aldrich). In order to confirm an effect of inhibiting gluconeogenesis according to composition ratios of GCG and IQC, the HepG2 cells were treated with Adiphenon complexes having various ratios (GCG: IQC=10:0˜0:10) at a total concentration of 100 μg/ml for 24 hours. Insulin (10 nM, Sigma Aldrich) known to inhibit gluconeogenesis was used as a positive control group. Thereafter, the cells were washed with Phosphate Buffered Saline (PBS; Sigma Aldrich), and then RNA was extracted using TaKaRa MiniBEST Universal RNA Extraction Kit (Takara Bio). cDNA was synthesized from the same amount (1 mg) of RNA using RevertAid 1^{st}-strand cDNA Synthesis Kit (Thermo Fisher Scientific). In order to confirm expression of PEPCK (Phosphoenol Pyruvate Carboxylkinase) and G6Pase (Glucose 6-Phosphatase), the rate-limit enzymes of gluconeogenesis, expression of the corresponding genes was observed using a CFX96 thermocycler (Bio-Rad) (FIG. 1).

As a result, as shown in FIG. 1, it was found that Adiphenon inhibited the expression of gluconeogenesis-related genes compared to the single substance treatment group regardless of their composition ratios, and that inhibition of the gene expression in Adiphenon was the best at a ratio of GCG:IQC=4:6 to 2.8. In other words, it was confirmed that Adiphenon can effectively inhibit gluconeogenesis in the liver cells.

### Example 2

### Effect of Promoting Glucose Transport in Fat Cells and Muscle Cells by Adiphenon Treatment

In order to identify another major mechanism of controlling blood sugar, it was confirmed whether Adiphenon could promote uptake of glucose in fat cells and muscle cells. Specifically, mouse-derived preadipocytes (3T3-L1) and myoblasts (C2Cl2) were purchased from ATCC. The 3T3-L1 cells were cultured in Dulbecco's Modified Eagle's Medium (DMEM; Sigma Aldrich) containing 10% bovine calf serum (Gibco) and 1% P/S, and the C2Cl2 cells were cultured in DMEM containing 10% FBS and 1% P/S. Differentiation into the fat cells was induced by the following method: The 3T3-L1 cells were cultured until a culture dish became 100% full, and then were treated in DMEM containing dexamethasone (1 mM; Sigma Aldrich), insulin (10 mg/ml; Sigma Aldrich), 3-isobutyl-1-methylxanthine (0.5 mM; Sigma Aldrich), FBS (10%), and P/S (1%) for 48 hours. Thereafter, the cells were cultured in DMEM containing 10% FBS, 10 mg/ml insulin, and 1% P/S every two days for 14 days to induce differentiation into complete fat cells. For differentiation into the muscle cells, the C2Cl2 cells were grown until a culture dish became 100% full, and then differentiated in DMEM medium containing 2% horse serum (HS; Gibco) and 1% P/S for 7 days. During the differentiation process, the medium was changed every day. In order to confirm transport of glucose in the differentiated fat cells and liver cells, starvation was induced the day before experiment with serum-free, low-glucose DMEM (Sigma Aldrich) containing only 1% P/S. Thereafter, Adiphenon of various composition ratios were treated for 24 hours according to the same method as that of Example 1, and experimental groups treated with insulin (100 nM) was additionally produced to confirm interaction with insulin. After 24 hours, an amount of glucose transported into the cells was quantified using a Glucose Uptake Assay kit (colorimetric, abcam). The quantification was performed using Tecan Infinite M200 Pro Microplate Reader (ab 412 nm).

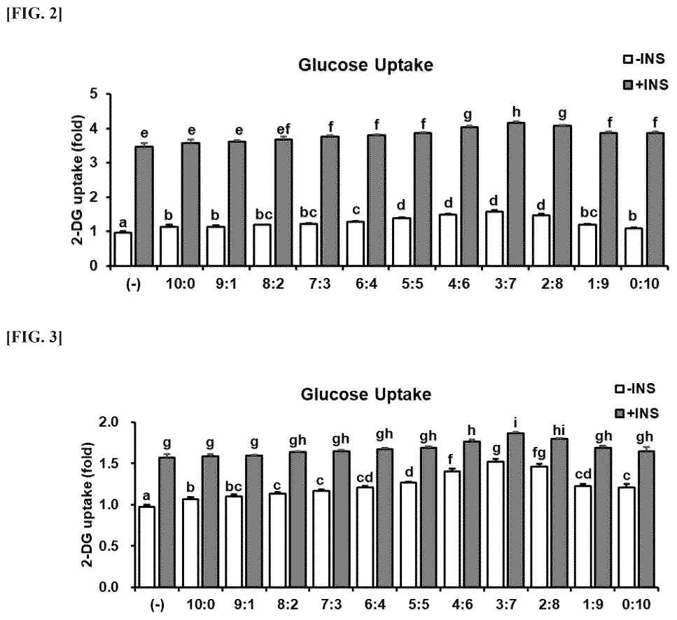

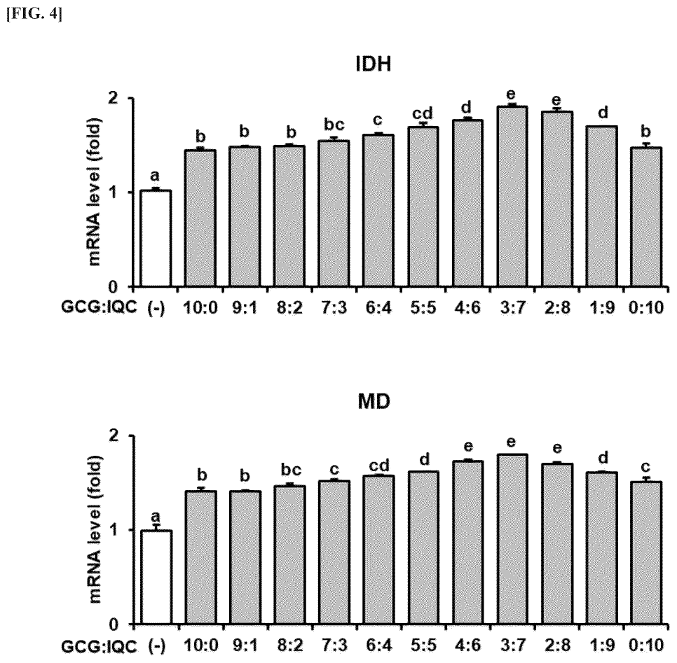

As a result, as shown in FIGS. 2 and 3, it was confirmed that Adiphenon of various composition ratios could effectively promote uptake of glucose in the fat cells and the muscle cells, and this effect was doubled in the presence of insulin. In addition, it was confirmed that an effect of transporting glucose by Adiphenon was most excellent at a ratio of GCG:IQC=4:6 to 2:8.

### Example 3

### Activation of Citric Acid Cycle in Cells by Adiphenon Treatment

Glucose introduced into cells may be either used as an energy source, or stored after being changed in the form of glycogen or fatty acids depending on the cells. In particular, if glucose is stored as fatty acids/neutral lipids in the fat cells, it can cause obesity due to increase in the body fat, so there is a need to check whether the absorbed glucose is used as an energy source. Glucose is decomposed into two molecules of pyruvic acid through glycolysis, and the pyruvic acid is converted to acetyl coenzyme A through further metabolism. The acetyl coenzyme A is used to synthesize ATP in the mitochondrial electron transport system through a citric acid cycle.

According to this, in order to see whether Adiphenon treatment can activate energy metabolism in the cells, after fat cells and muscle cells were differentiated in the same method as that of Example 2, they were treated with various composition ratios of Adiphenon for 24 hours. Thereafter, RNA was extracted and cDNA was synthesized in the same method as that of Example 1, and then expression of genes constituting the citric acid cycle was confirmed.

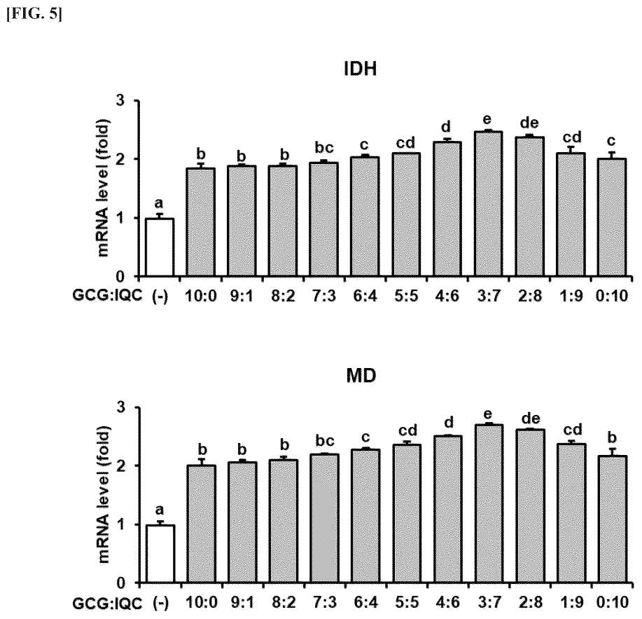

As a result, as shown in FIGS. 4 and 5, an effect of promoting expression of the genes constituting the citric acid cycle by treatment with various composition ratios of Adiphenon was identified in both of the fat cells (FIG. 4) and the muscle cells (FIG. 5). In addition, it was confirmed that the above effect by Adiphenon was most excellent at GCG:IQC composition ratios of 3:7 to 2:8.

Meanwhile, even though expression of the genes constituting the citric acid cycle increases, the citric acid cycle cannot operate smoothly if there is not enough oxygen or mitochondria in the cells. Under this situation, the pyruvic acid produces a small amount of ATP through a lactic acid fermentation process, instead of producing acetyl coenzyme A, and during this process, the lactic acid accumulates, which causes a fatigue in the cells. Accordingly, an aerobic energy metabolism activity in the cells can be indirectly confirmed by comparing a degree of lactic acid accumulation in the cells. Treatment of the cells with CoCl₂ induces a stabilization of HIF-1α, which causes the cells to think they are suffering from hypoxia. In this case, the cells recognize (mistakenly) that they are lacking oxygen and produce ATP using the lactic acid fermentation that does not consume oxygen, without being subjected to the oxidative phosphorylation process with mitochondria.

According to this, after the differentiated fat cells and muscle cells were treated with CoCl₂ (100 μM; Sigma Aldrich) for 12 hours to induce hypoxia, they were treated for 24 hours with Adiphenon of GCG:IQC=3:7 and 2:8 ratios that were confirmed to have the best synergistic effect in the field of energy metabolism, and an amount of lactic acid accumulated in the cells was measured (L-lactate Assay Kit (Colorimetric), abcam).

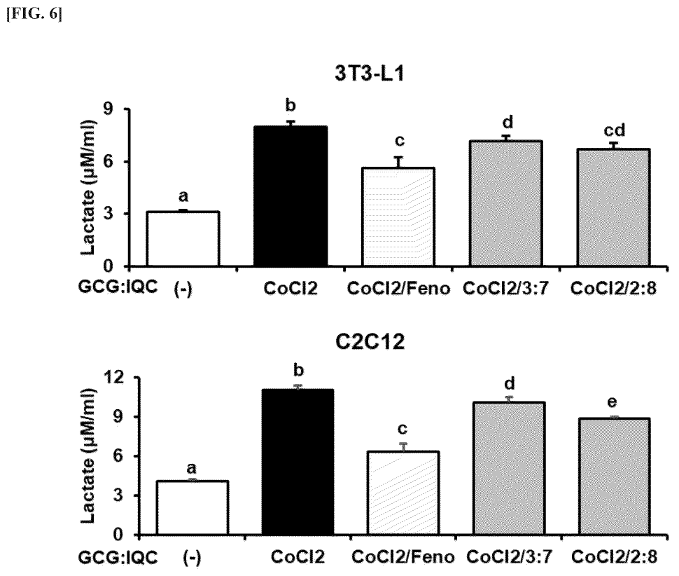

As a result, as shown in FIG. 6, the experimental result showing that Adiphenon inhibits lactic acid accumulation in the cells in addition to promoting gene expression of the citric acid cycle indicates that Adiphenon can promote aerobic energy metabolism in the cells.

### Example 4

### Comparison of Effects Depending on the Ratios of Adiphenon

In order to find an optimal ratio that shows the best synergy for each of composition ratios of Adiphenon, the experiments of Examples 1 and 2 were repeated after Adiphenon was treated at various composition ratios within the range of GCG:IQC=4:6 (about 1:1.5) to 2:8 (about 1:4).

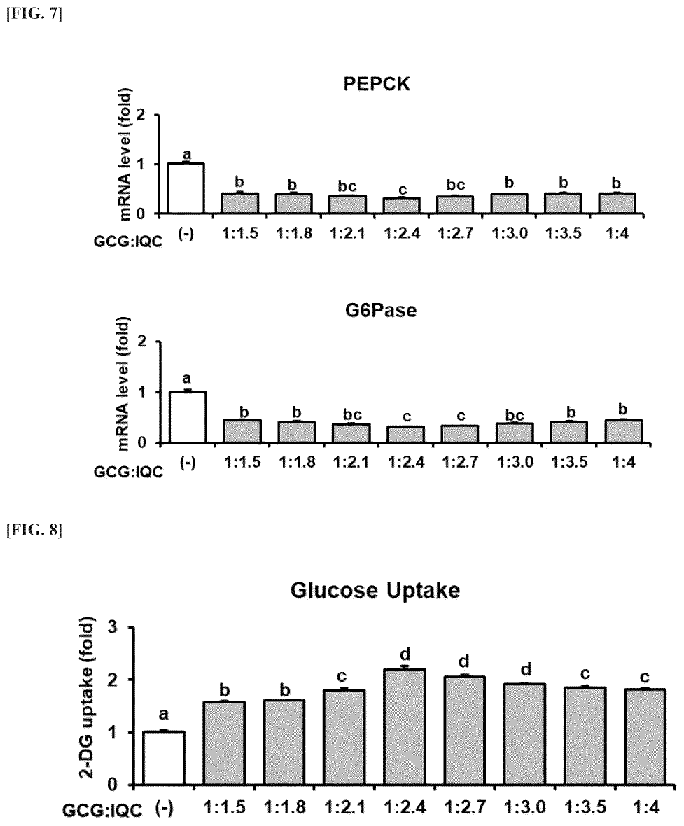

As a result, as shown in FIGS. 7 to 9, it was confirmed that the best synergy effect was observed when the ratios of GCG:IQC were between 1:2.1 and 1:2.7, more specifically at the ratio of 1:2.4.

What is claimed is:

1. A method for controlling blood sugar, comprising administering an effective amount of Gallocatechin Gallate (GCG) and Isoquercitrin (IQC) to a subject in need thereof.

2. The method according to claim 1, wherein Gallocatechin Gallate (GCG) and Isoquercitrin (IQC) are contained in a weight ratio of 9:1 to 1:9.

3. The method according to claim 1, wherein the subject needs to decrease gluconeogenesis in liver cells.

4. The method according to claim 1, wherein the subject needs to reduce expression of gluconeogenesis-related genes.

5. The method according to claim 1, wherein the subject needs to increase uptake and/or transport of glucose in fat cells or liver cells.

6. The method according to claim 1, wherein the subject needs to increase expression of citric acid cycle-constituting genes.

7. The method according to claim 1, wherein the subject needs to inhibit accumulation of lactic acids in cells.

8. The method according to claim 1, wherein the subject needs to promote aerobic energy metabolism in cells.

9. The method according to claim 1, wherein the method is a method for preventing, improving or treating diabetes, diabetic complications or sugar metabolism diseases.
